(19) United States
(12) Patent Application Publication
(10) Pub. No.: US 2006/0165797 A1
(43) Pub. Date: Jul. 27, 2006

(54) Dosage form for treating gastrointestinal disorders

(75) Inventor: John R. Plachetka, Chapel Hill, NC (US)

Correspondence Address: FITCH, EVEN, TABIN & FLANNERY, P. O. BOX 65973, WASHINGTON, DC, 20035, US

(73) Assignee: POZEN Inc., Chapel Hill, NC (US)

(21) Appl. No.: 11/328,259

(22) Filed: Jan. 10, 2006

Related U.S. Application Data
(60) Provisional application No. 60/643,137, filed on Jan. 12, 2005.

(51) Int. Cl. A61K 31/4439; A61K 9/24

(52) U.S. Cl. 424/472; 514/338

(57) ABSTRACT

The present invention is directed to drug dosage forms that can be used to treat diseases characterized by abnormal gastric acid secretion. The dosage forms have a core containing a proton pump inhibitor surrounded by an enteric coating or multiple particles containing proton pump inhibitor, each particle being surrounded by an enteric coating. The enteric coating delays the release of drug until the surrounding pH has risen. The tablets also include an outer coating that contains either a proton pump inhibitor or an H2 blocker. The outer coating is designed to rapidly dissolve in a patient's stomach.

### CROSS REFERENCE TO RELATED APPLICATIONS

The present application claims priority to, and the benefit of, U.S. provisional application 60/643,137, filed on Jan. 12, 2005. The contents of this prior application are hereby incorporated by reference in its entirety.

### FIELD OF THE INVENTION

The present invention is directed to pharmaceutical compositions for the treatment of gastrointestinal disorders, and particularly for the treatment of gastroesophogeal reflux disease. The pharmaceutical compositions contain a core or a plurality of particles with a therapeutically effective amount of a proton pump inhibitor (ppi). The core, or each particle, is surrounded by a coating that delays the release of drug. In addition, the compositions have a separate outer coating that contains either a proton pump inhibitor or an H2 blocker and which is designed to release drug immediately after ingestion by a patient.

### BACKGROUND OF THE INVENTION

Gastroesophogeal reflux disease (GERD) is a common disorder which, if untreated, can have serious health consequences. Symptoms of esophageal reflux can be acute, commonly arising from a provocative meal or recumbent posture in susceptible individuals. In GERD, symptoms of esophageal reflux are chronic, occurring throughout the day, frequently without any provocative cause. Effective treatment of GERD requires management of both acute and chronic reflux. Two types of agents frequently prescribed for the treatment of GERD are H2 blockers and proton pump inhibitors. H2 blockers prevent interaction between gastric cells that produce acid and histamine, an agent known to stimulate acid secretion. These drugs have a relatively rapid onset of action but a short duration of effectiveness (typically 8-12 hours). Examples of H2 blockers currently on the market are: cimetidine (Tagamet®); famotidine (Pepcid®); nizatidine (Axid®); and ranitidine (Zantac®). Unfortunately, many patients with more severe forms of GERD do not get adequate relief from these H2 blockers.

Proton pump inhibitors (ppis) are typically prescribed for GERD patients that cannot be effectively treated with H2 blockers alone. PPIs bind to and inhibit the cellular enzyme responsible for secreting acid into the stomach. These drugs are more effective than H2 blockers at reducing acid secretion and typically have a duration of action long enough that they only need to be taken once a day. This has made the proton pump inhibitors of interest in therapies designed to provide long-term protection to the stomach by maintaining an elevated pH (see published U.S. application 2003069255; and WO 2004/060372). Examples of proton pump inhibitors currently on the market are: omeprazole (Prilosec®); esomeprazole (Nexium®); lansoprazole (Prevacid®); pantoprazole (Protonix®); rabeprazole (Aciphex®).

Because proton pump inhibitors are typically acid labile, they have usually been formulated as tablets with an enteric coating (see U.S. Pat. No. 4,853,230; see also: U.S. Pat. No. 4,786,505; EP 0277,741; and EP 0342,522) and this may contribute to a slow onset of effectiveness. Patients usually do not get substantial relief from their symptoms for at least 24 hours after ingestion of a tablet and several days may be required (Clin. Pharmakinet 20:38-49(1991)). Recently, attempts have been made to reduce the time needed for achieving a therapeutic effect by using dosage forms which include an antacid buffer, instead of an enteric coating, to protect against acid degradation (U.S. Pat. Nos. 5,840,737; 6,489,346; 6,645,988; 6,780,882; 4,786,505; and 6,183,776).

More recently, a newer class of proton pump inhibitor which competes with potassium at the acid pump, has been developed. This class of compound has been referred to variably as “reversible proton pump inhibitors” and “acid pump antagonists.” Examples include AZD-0865, AR-H047108, CS-526, pumaprazole, revaprazan and soraprazan (see WO9605177 and WO9605199). Not all of the compounds in this newer class of proton pump inhibitor is acid labile.

### SUMMARY OF THE INVENTION

Ideally, a dosage form should provide for both the rapid relief of patient symptoms and for long term effectiveness to prevent a recurrence of symptoms. The present invention is based upon the development of tablet dosage forms that provide for a multi-phase release of acid inhibitor. In one embodiment, the tablets have an outer coating or an immediate release component that quickly dissolves in the stomach of a patient immediately after ingestion (within 60 minutes and preferably within 15 minutes) and which releases either an H2 blocker or a proton pump inhibitor. Although proton pump inhibitors may be acid labile, a sufficient amount can be incorporated into the immediate release component or outer coatings to inhibit the production of stomach acid. Oral dosage forms also include a core or a delayed release component that may be enterically coated and which contains a proton pump inhibitor. An enteric coating should be present in all dosage forms in which the core contains an acid labile proton pump inhibitor and may, or may not, be present in cases where the core contains a non-acid labile proton pump inhibitor, i.e., an inhibitor that is stable at a pH of 1.0-3.0. The enteric coating prevents the release of inhibitor from the core until the pH of the stomach has risen or this component of the dosage form has entered a patient's intestine. Thus, an acid labile proton pump inhibitor is protected from degradation and, as a result, a higher percentage will eventually enter a patient's blood stream and provide long-term relief of symptoms.

In an alternative design, there may be numerous particles of enterically coated proton pump inhibitor within a tablet or capsule. As discussed above, the enteric coating prevents the release of drug until the pH of the surrounding medium is at least 3.5 and may, if desired, also provide for the timed release of drug. This coating should be present for all acid labile proton pump inhibitors but is not necessarily enteric in cases where the in the core ppi is not acid labile. Sufficient proton pump inhibitor is contained within the particle cores so that, following release, gastric pH rises to or is maintained at 3.5 or higher. Each particle may include an outer coating that surrounds the enteric coating or the tablet or capsule may have one or more outer coatings that surround a plurality of particles. It will be understood that, when used in the context of a dosage form with numerous particles, the term “outer coating” may refer to either a single coating or to multiple coatings. The outer coating is not itself enteric and is not surrounded by a separate enteric coating. If desired, one or more layers of material (e.g., containing excipients or active ingredients) may be included between the particles containing proton pump inhibitor and the outer coating. Upon ingestion by a patient, sufficient acid inhibitor is released from the outer coating into a patient's stomach within 60 minutes, and preferably within 15 minutes, to prevent reduction in gastric pH by production of new stomach acid. The invention also includes methods of making these dosage forms and methods in which they are used to treat patients.

In its first aspect, the invention is directed to a pharmaceutical composition in the form of a tablet with an enterically coated core or a pharmaceutical composition with a plurality of particles each of which is surrounded by an enteric coating. The core (or the particles) contains a therapeutically effective amount of a proton pump inhibitor. The term “therapeutically effective” indicates a sufficient amount of drug to alleviate a patient's symptoms by inhibiting acid production in the stomach, resulting in an increased median gastric pH, where median gastric pH is the median of gastric pH measurements taken at regular intervals over a 24 hour period. Preferably, after 24 hours median gastric pH should be at least about 3.5 and, more preferably, at least 4.5. The enteric coating that surrounds the core or particles is designed to prevent the release of proton pump inhibitor until the surrounding pH is at least 3.5, with pH values greater than 5.5 being preferred. In some cases, the elevated pH may not be obtained until the enterically coated drug reaches the patient's intestine, particularly the first time that a tablet is taken. However, in patients taking medication on a daily basis, the stomach should soon stabilize at a higher pH. In these patients, the enterically coated component will rapidly release proton pump inhibitor into the stomach, preferably within 60 minutes after ingestion. Alternatively, a coating may be used which delays release of the proton pump inhibitor by some mechanism other than gastric pH e.g., in a time dependent manner.

In addition to having an enterically coated component, the dosage forms described above will include an outer coating containing a therapeutically effective amount of either a proton pump inhibitor or an H2 blocker. Drug in the outer coating is not enterically coated and should be released into a patient's stomach immediately after ingestion. In dosage forms where the outer coating contains an acid labile proton pump inhibitor, it is recognized that a significant portion of the dosage delivered may be degraded in stomach acid before it can be absorbed into a patient's bloodstream. Nevertheless, sufficient inhibitor is taken up to provide for an alleviation of symptoms associated with GERD. In general, enough drug, i.e., proton pump inhibitor or H2 blocker, should be present to significantly suppress gastric acid secretion within six hours after ingestion by a patient.

Preferably, solid oral dosage forms will comprise an enterically coated core of proton pump inhibitor with non-enterically coated proton pump inhibitor or H2 blocker on the outside of the core in a film coat. This outer film coat should be thin to provide for essentially immediate drug release. Its thickness should generally be no more than 1,000 microns and preferably, it should be between 25 and 500 microns. Coatings with these characteristics can be obtained by spraying enterically coated cores with a film-forming solution containing drug. In addition to proton pump inhibitors or H2 blockers, the outer coating may contain other agents such as stabilizers, buffers or alkaline substances. When buffers or alkaline substances are used, they should be designed to raise the pH of the stomach.

Unless otherwise indicated, the term “proton pump inhibitor” or “ppi” as used herein includes the reversible proton pump inhibitors or acid pump antagonists such as AZD-0865, AR-H047108, CS-526, pumaprazole, revaprazan and soraprazan. Preferred proton pump inhibitors for use in both the outer coating and inner core of tablets include omeprazole, esomeprazole, lansoprazole, pantoprazole and rabeprazole. All of these drugs are commercially available or can be synthesized using techniques well known in the art. They should preferably be present at a dosage of 1-200 mg, and more preferably at 5-100 mg in the outer coating and at 5-600 mg in the inner core. Examples of preferred dosages for particular proton pump inhibitors in both the outer coating and inner core are: 5-50 mg omeprazole; 5-100 mg esomeprazole; 15-150 mg lansoprazole; 10-200 mg pantoprazole; and 5-100 mg rabeprazole. Other proton pump inhibitors may also be used, including pariprazole and leminoprazole.

Preferred H2 blockers for use in the outer coating of tablets include: cimetidine; ranitidine; famotidine; ebrotidine; pabutidine; lafutidine; and nizatidine. These drugs should preferably be present at 1-300 mg and more preferably at 5-150 mg.

The invention also includes methods of treating a patient for a disease or condition characterized by abnormal gastric acid production, gastric acid reflux, or damage to the gastrointestinal tract, by administering one or more of the tablets described above. Specific diseases or conditions that may be treated include: duodenal ulcers; gastric ulcers; gastroesophageal reflux disease; severe erosive esophagitis; poorly responsive systemic GERD; and Zollinger Ellison syndrome.

The invention also includes methods for manufacturing unit dosage forms having the characteristics described above. These methods involve first forming a core comprising 5 to 600 mg of a proton pump inhibitor. A coating, preferably an enteric one that does not dissolve until the surrounding pH is at least 3.5, is then applied to the core. Optionally, one or more additional layers are applied over the coating surrounding the core. These additional layers may contain drugs, excipients, buffers or alkaline agents. Finally, there is an outer coating that is applied by spraying. This outer coating is not enteric and contains 1-200 mg of a proton pump inhibitor or 1-300 mg of a H2 blocker. Preferably, the outer coating is no more than 1,000 microns in diameter, and more preferably, it should be between 25 and 500 microns in diameter Optionally, a stabilizer or a buffer designed to raise the pH of the stomach may be included in the outer coating or as a separate layer immediately below the coating. The most preferred proton pump inhibitors for use in tablets and the most preferred H2 blockers are those that are described above.

### BRIEF DESCRIPTION OF THE DRAWINGS

FIG. 1: Omeprazole Pellets: FIG. 1 is a schematic drawing showing both uncoated pellets of omeprazole (A) and delayed release formulations in which a core pellet (A) is surrounded by a subcoating (B) and an enteric coating (C). The composition of the core pellet and of each of the outer layers is provided in Example 1. The pellets may be either compressed into a tablet or loaded into a capsule.

FIG. 2: Tablets Containing Immediate Release and Delayed Release Omeprazole Pellets: The figure shows a compressed tablet containing both uncoated omeprazole pellets (D), which release drug immediately upon exposure to gastric fluid, and enterically coated omeprazole pellets (E), that delay drug release until the pH of the surrounding medium is at least 3.5. Optionally, the tablets may be coated with a non-enteric film that dissolves immediately after ingestion. The tablets are described in detail in Example 2.

FIG. 3: Bilayer Tablet: FIG. 3 shows a bilayer tablet containing enterically coated, delayed release omeprazole pellets (F) and immediate release omeprazole granules (G). For a fuller description, see Example 3.

FIG. 4: Multilayer Tablet Dosage Form: FIG. 4 shows a tablet having a core layer (H) containing lansoprazole. This is surrounded by a barrier coating layer (I) that serves to protect the core. A third layer (J) is an enteric coating that does not dissolve until the surrounding medium is at a pH of 3.5 or higher. Finally the outermost layer (K) is a film coating that contains a therapeutically effective amount of lansoprazole. This outer film is not enteric and releases the lansoprazole immediately after ingestion. The exact components present in each layer are described in Example 4.

### DETAILED DESCRIPTION OF THE INVENTION

The present invention is directed to a dosage form that provides for both the quick release of an acid inhibitor to reduce anticipated reflux symptoms in a patient, as well as for the delayed release of a proton pump inhibitor to provide for longer term relief and to prevent the return of symptoms. The active ingredients used in tablets, i.e., proton pump inhibitors and H2 blockers, are well known in the art and the preferred agents described above are commercially available. If desired, drugs can also be manufactured using methodology well known in the art.

Making of Pharmaceutical Preparations

The tablets of the present invention can be made in accordance with methods that are standard in the art (see e.g., Remington's Pharmaceutical Sciences, 16^{th} edition, A. Oslow, editor, Easton, Pa. (1980)). Drugs may be prepared in admixture with conventional excipients, carriers, buffers, flavoring agents, etc. Typical carriers include, but are not limited to: water; salt solutions; alcohols; gum arabic; vegetable oils; benzyl alcohols; polyethylene glycols; gelatin; carbohydrates, such as lactose, amylose or starch; magnesium stearate; talc; silicic acid; paraffin; perfume oil; fatty acid esters; hydroxymethylcellulose; polyvinyl pyrrolidone; etc. Pharmaceutical preparations can be sterilized and, if desired, mixed with auxiliary agents such as: lubricants; preservatives; disintegrants; stabilizers such as cyclodextrans; wetting agents; emulsifiers; salts; buffers; coloring agents; flavoring agents; or aromatic substances. Particularly preferred are buffers that can raise the pH of the stomach. For example bicarbonate buffers may be included in the outer coating or as a rapidly dissolving, separate layer immediately below the outer coating.

The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups. Enteric coating layers may also contain pharmaceutical plasticizers such as: triethyl citrate; dibutyl phthalate; triacetin; polyethylene glycols; polysorbates; etc. Additives such as dispersants, colorants, anti-adhering and anti-foaming agents may also be included.

Making of Tablet Dosage Forms

Tablets can be made using standard technology well known in the art. Drugs used in the core or the outer coating may be granulated by methods such as slugging, low-shear or high-shear granulation, wet granulation, or fluidized bed granulation. Outer coatings may be formed by preparing a mixture containing appropriate polymers and a sufficient amount of drug to produce a therapeutically effective dose. The solution may then be sprayed on preformed, enterically-coated cores to produce the final tablets. If desired, a buffer layer or layer containing other agents may be interspersed between the enterically coated core and the outer coating.

Treatment of Patients

The pharmaceutical compositions described above can be used to treat a patient for any disease or condition in which proton pump inhibitors are indicated. The most common condition will be GERD. Other conditions include duodenal ulcers, gastric ulcers, severe erosive esophagitis, and Zollinger Ellison syndrome In all cases, a patient should be administered a sufficient daily dosage to eliminate the symptoms associated with excess gastric acid production. Typical daily dosages of all of the preferred agents are well known in the art. In general, anywhere from 5-600 mg of proton pump inhibitor may be administered in the core of tablets and an additional 1-200 mg in the outer coating. When an H2 blocker is used in the outer coating, it should generally be administered at a dosage of from 1 to 300 mg. The final dosages used will be selected by the attending physician based upon clinical conditions and using methods well known in the art. As a general rule, drugs will be designed to be taken once a day but other dosing regimens may also be used. In particular, under some conditions twice daily doses may be preferred. In general, it is desirable to administer the drugs before anticipated onset of symptoms due to eating, drinking, or any other provocative activity. Treatments should be continued even after symptoms associated with excess acid production have subsided and it is expected that patients will typically continue taking pharmaceutical compositions for many years.

### EXAMPLES

### Example 1

### Omeprazole Delayed Release and Immediate Release Capsule

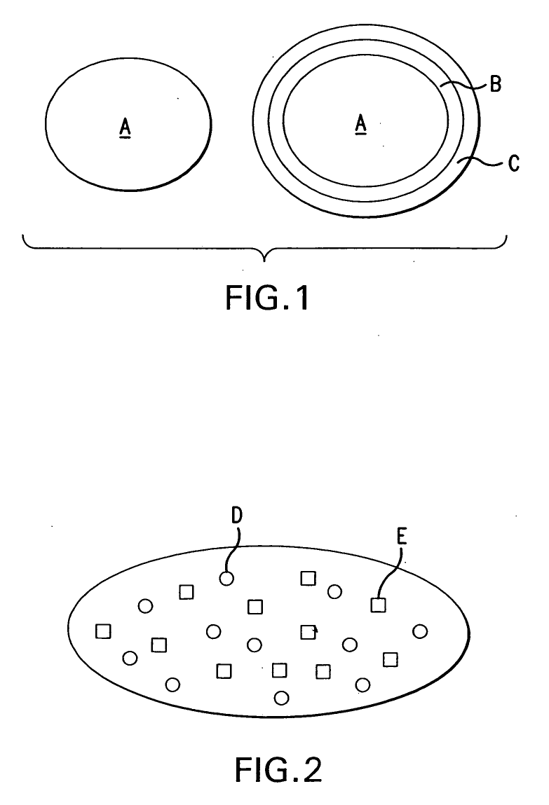

The present example is directed to a capsule that contains omeprazole pellets with (delayed release) and without (immediate release) an enteric coat (see FIG. 1 for schematic of pellets). The omeprazole pellets contain sodium bicarbonate as an alkalizing excipient. Other soluble alkalizing agents that could be used include potassium bicarbonate, sodium carbonate, sodium hydroxide, and combinations of these agents. The alkalizing agent helps solubilize and protect omeprazole from degradation before it is absorbed. Sodium lauryl sulfate is present in pellets to help in the wetting of omeprazole. Other surfactants could be used to perform the same function. In this example, hydroxypropylmethylcellulose is present to help in granule formation, and sodium starch glycolate is included as a disintegrant. Other excipients may also be used to perform these functions. The pellets are prepared by the wet massing technique and conventional extrusion and spheronization processes.

After pellets are formed they are dried and classified according to size. Pellets for delayed release are first coated with a protective subcoating containing povidone. Other coating ingredients that could be used include Opaspray K-1-4210A™ and Opadry YS-1-7006™ (trademarks of Colorcon, West Point, Pa.). Polymer film coating ingredients such as hydroxypropylmethylcellulose 2910 and polyethylene glycol 8000 in a subcoating suspension could also be used. Other ingredients that may be present in subcoatings include: plasticizers such as triethyl citrate or dibutyl phthalate; anti-adhering agents such as talc; lubricating ingredients such as magnesium stearate; and opacifiers such as titanium dioxide.

The subcoated pellets are enteric coated using enteric coating polymers. In the present example, the enteric coating polymer is methacrylic acid copolymer and the plasticizer is diethyl phthalate which is dissolved in a mixture of acetone and alcohol. The enteric film does not dissolve in the acidic pH normally found in the stomach but dissolves when the pH is above pH 4.5. Other materials that can be used in enteric coatings include: hydroxypropyl methylcellulose phthalate; ammoniomethacrylate copolymer; shellac; polyvinyl acetate phthalate; and cellulose acetate phthalate.

A. Preparation of Omeprazole Pellets

Hydroxypropylmethylcellulose is dissolved in water and sodium lauryl sulfate is then added to the solution. Omeprazole, microcrystalline cellulose, sodium bicarbonate and sodium starch glycolate are dry mixed together and granulated with the granulating solution. The wet mass is mixed until a proper consistency is reached. It is then pressed through an extruder and spheronized to form pellets. The pellets are then dried and classified into suitable particle size ranges.

TABLE 1
Composition of Omeprazole Pellets

| | % W/W | mg/capsule |
|---|---|---|
| Omeprazole, USP | 28.57 | 20.0 |
| Sodium bicarbonate, USP | 28.57 | 20.0 |
| Microcrystalline cellulose, NF | 33.57 | 23.5 |
| Hydroxypropylmethylcellulose, USP | 4.29 | 3.0 |
| Sodium lauryl sulfate, NF | 0.71 | 0.5 |
| Sodium starch glycolate, NF | 4.29 | 3.0 |
| Total | 100 | 70 |

B. Subcoating

Half of the pellet cores described above are coated using povidone solution to a weight gain of 1-2%.

TABLE 2
Subcoating Solution

| | % W/W |
|---|---|
| Povidone (K29-32), USP | 10.00 |
| Alcohol, USP | 90.00 |
| Total | 100.00 |

C. Enteric Coating

Eudragit L-100 is dissolved in isopropanol and acetone and diethyl phthalate is then dissolved. The solution is sprayed on the subcoated pellet cores using film coating equipment. A sample of the pellets is tested for gastric resistance before stopping the coating process.

TABLE 3
Enteric Spray Coating Composition

| | % W/W |
|---|---|
| Methacrylic Acid Copolymer, NF | 8.20 |
| (Eudragit L-100) | |
| Diethyl Phthalate, NF | 1.70 |
| Acetone, NF | 33.30 |
| Isopropyl Alcohol, USP | 56.80 |
| Total | 100.0 |

D. Preparation of Capsules

Omeprazole immediate release pellets (uncoated) and delayed release pellets (enteric coated) are blended together and used to fill capsules to contain 20 mg delayed release omeprazole and 20 mg immediate release omeprazole per capsule.

### Example 2

### Omeprazole Delayed Release and Immediate Release Tablet

This tablet is compressed from a mixture of enteric coated omeprazole pellets and immediate release pellets and is illustrated in FIG. 2. The formulation of omeprazole pellets contains 30 mg omeprazole and uses mannitol as a filler, hydroxypropylcellulose as a binder and microcrystalline cellulose as a disintegrant and filler. Delayed release pellets are coated with a subcoating followed by enteric coating with an aqueous dispersion of methacrylic acid copolymer.

A. Formation of Omeprazole Pellets

Omeprazole, mannitol, microcrystalline cellulose, hydroxypropylcellulose, sodium lauryl sulfate and dibasic sodium phosphate are dry mixed together and granulated with purified water. The wet mass is mixed until a proper consistency is reached. It is then pressed through an extruder and spheronized to form pellets. The resulting pellets are dried and classified into suitable particle size range. The composition of the pellets is shown in Table 4.

TABLE 4
Composition of Omeprazole Pellets

| | % W/W | mg/tablet |
|---|---|---|
| Omeprazole, USP | 26.1 | 30.0 |
| Mannitol, USP | 52.2 | 60.0 |
| Microcrystalline cellulose, NF | 13.9 | 16.0 |
| Hydroxypropyl cellulose, USP | 4.6 | 5.25 |
| Sodium lauryl sulfate, NF | 0.65 | 0.75 |
| Dibasic sodium phosphate, USP | 2.6 | 3.0 |
| Total | 100 | 115 |

B. Barrier Coating

Opadry clear is added slowly to purified water and the preparation is mixed until the Opadry is fully dispersed. The solution is sprayed on to half of the omeprazole pellets formed as described above until 1-2% of Opadry clear is deposited on the pellets.

TABLE 5
Subcoating Solution

| | % W/W |
|---|---|
| Opadry ® Clear YS-1-7006 | 5.00 |
| Purified Water, USP | 95.00 |
| Total | 100.00 |

C. Enteric Coating

Eudragit L30D is dispersed in a purified water and simethicone emulsion. Talc and triethyl citrate are then dispersed. The suspension is sprayed on the pellet cores which contain the barrier film coat using film coating equipment. A sample of the pellets is tested for gastric resistance before stopping coating process.

TABLE 6
Enteric Coating Spray Composition

| | % W/W |
|---|---|
| Methacrylic Acid Copolymer, NF | 15.60 |
| (Eudragit L30D 30% dispersion) | |
| Talc, USP | 7.60 |
| Triethyl citrate, NF | 1.60 |
| Simethicone Emulsion, USP | 0.20 |
| Purified Water, USP | 74.80 |
| Total | 100.0 |

D. Formation of Tablets

Omeprazole delayed release pellets and immediate release pellets are blended together with magnesium stearate and microcrystalline cellulose and compressed into a tablet containing 30 mg delayed release omeprazole and 30 mg immediate release omeprazole. The tablet can be film coated with pigmented Opadry or an equivalent containing typical film coating ingredients including hydroxypropyl methylcellulose, polyethylene glycol and colorant.

### Example 3

### Bilayer Film Coated Tablet with Delayed Release

### Omeprazole and Immediate Release Omeprazole

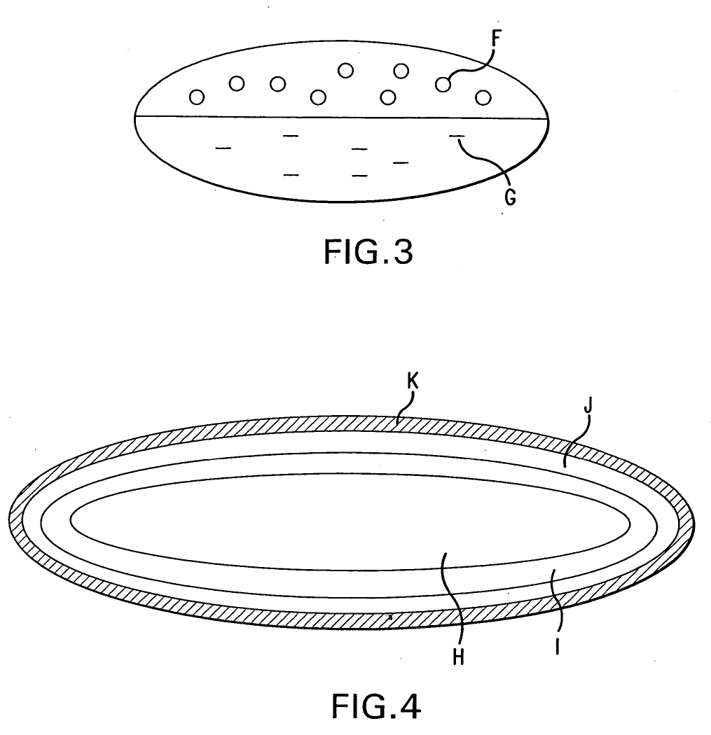

The bilayer tablet of the present example is compressed from enteric coated pellets and omeprazole granules and is illustrated in FIG. 3. Enteric coated omeprazole pellets can be prepared as described in Example 1 or 2. Omeprazole granules are prepared using povidone as a binder, microcrystalline cellulose as a filler and disintegrant and mannitol as a filler.

A. Formation of Omeprazole Granules

Omeprazole, microcrystalline cellulose, povidone, sodium lauryl sulfate, and dibasic sodium phosphate are mixed in a granulator. Water is added and mixed until a suitable granule is formed. The granules are dried in an oven and milled. The milled granules are blended with magnesium stearate and microcrystalline cellulose.

TABLE 7
Composition of Omeprazole Granules

| | % W/W | mg/tablet |
|---|---|---|
| Omeprazole, USP | 12.5 | 10.0 |
| Microcrystalline cellulose, NF | 37.5 | 30.0 |
| Mannitol, USP | 37.5 | 30.0 |
| Povidone, USP | 6.25 | 5.0 |
| Sodium lauryl sulfate, NF | 0.94 | 0.75 |
| Dibasic sodium phosphate, USP | 4.31 | 3.45 |
| Magnesium stearate, NF | 1.0 | 0.8 |
| Total | 100 | 80 |

B. Formation of Tablets

The enteric coated pellets are mixed with microcrystalline cellulose and magnesium stearate. The blend consisting of enteric coated omeprazole pellets and the blend consisting of omeprazole granules are compressed into a bilayer tablet using a bilayer tablet press. The tablet can be film coated with pigmented Opadry or equivalent containing typical film coating ingredients including hydroxypropyl methylcellulose, polyethylene glycol and colorant.

### Example 4

### Delayed Release Lansoprazole Core and Lansoprazole

### Immediate Release in Film Coat

A schematic diagram of a four layer tablet dosage form is shown in FIG. 4. The first layer (H) is a core containing lansoprazole distributed throughout a matrix of pharmaceutically acceptable fillers, excipients, binding agents, disintegrants, and lubricants.

The second layer (I) is a barrier layer which protects the first layer containing lansoprazole. The barrier film coat is applied by conventional pan coating technology and the weight of the barrier coat may vary from 1% to 3% of the core tablet weight. In particular embodiments, the core tablet is coated with coating ingredients such as Opaspray® K-1-4210A or Opadry® YS-1-7006 (Colorcon, West Point, Pa.). Polymer film coating ingredients such as hydroxypropylmethylcellulose 2910 and polyethylene glycol 8000 in a coating suspension may also be used.

The third layer (J) is an enteric film coat. In this example, hydroxypropyl methylcellulose phthalate is the enteric coating ingredient, cetyl alcohol is a plasticizer and acetone and alcohol are solvents. Other materials for enteric coating are ammonio methacrylate copolymer, shellac, polyvinyl acetate phthalate, hydroxypropyl methylcellulose trimellitate and cellulose acetate phthalate.

The fourth layer (K) is a film coating containing lansoprazole in an effective amount which is released from the dosage form as soon as the film coat dissolves. The film coat is applied by conventional pan coating technology and may vary from 4% to 8% of the core tablet weight, depending upon the amount of drug to be applied. Other ingredients are, plasticizers such as triethyl citrate, dibutyl phthalate, anti-adhering agents such as talc, lubricating ingredients such as magnesium stearate, opacifiers such as, titanium dioxide, and ammonium hydroxide to adjust the pH of the dispersion. The film coating is thin and rapidly releases lansoprazole for absorption. Therefore, 10 mg lansoprazole releases first and then the core erodes and releases 15 mg lansoprazole.

A. Preparation of Lansoprazole Core Layer (H)

Lansoprazole, microcrystalline cellulose, lactose, povidone, sodium lauryl sulfate, and dibasic sodium phosphate are dry mixed and wet granulated in a granulator with sufficient purified water. The wet granules are dried, milled, and blended with microcrystalline cellulose and magnesium stearate. The final granule blend is compressed into tablets.

TABLE 8
Composition of Lansoprazole Core

| | % W/W | mg/tablet |
|---|---|---|
| Lansoprazole, USP | 7.5 | 15.0 |
| Microcrystalline cellulose, NF | 30.0 | 60.0 |
| Lactose, NF | 55.0 | 110.0 |
| Povidone, USP | 3.0 | 6.0 |
| Sodium lauryl sulfate, NF | 1.0 | 2.0 |
| Dibasic sodium phosphate, USP | 2.5 | 5.0 |
| Magnesium Stearate, NF | 1.0 | 2.0 |
| Total | 100.00 | 200 |

B. Formation of Barrier Layer (I)

Opadry clear is added slowly to purified water and mixing is continued until it is fully dispersed. The solution is sprayed on to the tablet cores in a conventional coating pan until the desired amount of Opadry clear is deposited on the tablets.

TABLE 9
Barrier Layer Spray Solution

| | % W/W |
|---|---|
| Opadry ® Clear YS-1-7006 | 5.00 |
| Purified Water, USP | 95.00 |
| Total | 100.00 |

C. Formation of Enteric Coating

Hydroxypropylmethylcellulose phthalate and cetyl alcohol are dissolved in a mixture of alcohol and acetone. The solution is then sprayed on to the tablet bed using standard coating equipment. A sample of the tablets is tested for gastric resistance and the coating process is stopped if the tablets pass the test.

TABLE 10
Enteric Coating Spray Composition

| Enteric Coating Ingredients | % W/W |
|---|---|
| Hydroxypropylmethylcellulose phthalate, NF | 5.5 |
| Cetyl alcohol, NF | 0.3 |
| Acetone, NF | 66.3 |
| Alcohol, USP | 27.9 |
| Total | 100.00 |

D. Lansoprazole Film Coating

Lansoprazole is dispersed in purified water containing dibasic sodium phosphate and polysorbate 80. After thorough mixing, Opadry clear is added slowly and mixing is continued until the Opadry is fully dispersed. The suspension is sprayed on to the tablet cores in a conventional coating pan until the desired amount of lansoprazole is deposited on the tablets.

TABLE 11
Lansoprazole Film Coating Composition

| | % W/W |
|---|---|
| Lansoprazole, USP | 2.50 |
| Opadry ® Clear YS-1-7006 | 7.50 |
| Polysorbate 80, NF | 0.75 |
| Dibasic sodium phosphate, USP | 1.20 |
| Purified Water, USP | 88.05 |
| Total | 100.00 |

### Example 5

### Treatment Example

A patient is determined by his physician to be afflicted with GERD, which is provoked and aggravated by heavy meals before sleep. The patient is prescribed tablets as described in Example 4 to be taken each evening, 45 minutes before the evening meal. Untreated, the patient suffers from severe symptoms of esophageal reflux approximately 30 minutes after beginning dinner, and then throughout the night, requiring him to sleep seated in a recliner. After the patient takes the prescribed tablets the first evening, he experiences insignificant symptoms after dinner and is able to sleep lying in bed. On subsequent evenings, his GERD remains under control.

All references cited herein are fully incorporated herein by reference. Having now fully described the invention, it will be understood by those of skill in the art that the invention may be performed within a wide and equivalent range of conditions, parameters and the like, without affecting the spirit or scope of the invention or any embodiment thereof.

What is claimed is:

1. A pharmaceutical composition in the form of a tablet comprising:
a) an enterically coated core comprising a therapeutically effective amount of a proton pump inhibitor surrounded by an enteric coating, wherein said enteric coating does not release said proton pump inhibitor until the pH of the surrounding medium is at least 3.5; and
b) an outer coating surrounding said enterically coated core, wherein:
i) said outer coating comprises a sufficient amount of an acid inhibitor to suppress gastric acid secretion within 6 hours after ingestion by a patient, and wherein said acid inhibitor is selected from the group consisting of: a proton pump inhibitor and an H2 blocker;
ii) said outer coating is not an enteric coating, is not surrounded by an enteric coating, and releases said acid inhibitor within 60 minutes after ingestion.

2. The tablet of claim 1, wherein said outer coating:
a) has a thickness of less than 1000 microns; or
b) comprises a stabilizer or a buffer; or
c) both has a thickness of less than 1000 microns and comprises a stabilizer or a buffer.

3. The tablet of claim 1, wherein said acid inhibitor in said outer coating is a proton pump inhibitor at 1-200 mg and is selected from the group consisting of: omeprazole; esomeprazole; lansoprazole; pantoprazole; rabeprazole; AZD-0865, AR-H047108, CS-526, pumaprazole, revaprazan and soraprazan.

4. The pharmaceutical composition of claim 3, wherein said enterically coated core comprises 5-600 mg of a proton pump inhibitor selected from the group consisting of: omeprazole; esomeprazole; lansoprazole; pantoprazole; and rabeprazole.

5. The pharmaceutical composition of claim 4, wherein said proton pump inhibitor is selected from the group consisting of: omeprazole, present in said enterically coated core at between 5 mg and 50 mg; esomeprazole, present in said enterically coated core at 5-100 mg; lansoprazole, present in said enterically coated core at 15-150 mg; pantoprazole, present in said enterically coated core at between 10 mg and 200 mg; rabeprazole, present in said enterically coated core at between 5 mg and 100 mg.

6. The pharmaceutical composition of claim 1, wherein:
a) said acid inhibitor in said outer coating is a proton pump inhibitor present at 1-200 mg and selected from the group consisting of: omeprazole; esomeprazole; lansoprazole; pantoprazole; rabeprazole; AZD-0865; AR-H047108; CS-526; pumaprazole; revaprazan; and soraprazan;
b) said enterically coated core comprises 1-600 mg of a proton pump inhibitor is selected from the group consisting of: omeprazole; esomeprazole; lansoprazole; pantoprazole; and rabeprazole.

7. The tablet of claim 1, wherein said acid inhibitor in said outer coating is an H2 blocker.

8. The pharmaceutical composition of claim 7, wherein said acid inhibitor is present in said outer coating at 1-300 mg and is selected from the group consisting of: cimetidine; ranitidine; famotidine; ebrotidine; pabutidine; lafutidine; and nizatidine.

9. The pharmaceutical composition of claim 8, wherein said enterically coated core comprises 1-600 mg of a proton pump inhibitor is selected from the group consisting of: omeprazole; esomeprazole; lansoprazole; pantoprazole; and rabeprazole.

10. The pharmaceutical composition of claim 9, wherein said proton pump inhibitor is selected from the group consisting of: omeprazole, present in said enterically coated core at between 5 mg and 50 mg; esomeprazole, present in said enterically coated core at 5-100 mg; lansoprazole, present in said enterically coated core at 15-150 mg; pantoprazole, present in said enterically coated core at between 10 mg and 200 mg; and rabeprazole, present in said enterically coated core at between 5 mg and 100 mg.

11. The pharmaceutical composition of claim 1, wherein:
a) said acid inhibitor in said outer coating is an H2 blocker present at 1-300 mg and selected from the group consisting of: cimetidine; ranitidine; famotidine; ebrotidine; pabutidine; lafutidine; and nizatidine. and
b) said enterically coated core comprises 5-600 mg of a proton pump inhibitor, wherein said proton pump inhibitor is selected from the group consisting of: omeprazole, esomeprazole, lansoprazole, pantoprazole and rabeprazole.

12. A pharmaceutical composition in the form of a tablet or capsule comprising:
a) a plurality particles together comprising a therapeutically effective amount of a proton pump inhibitor wherein each particle is surrounded by an enteric coating that does not release said proton pump inhibitor until the pH of the surrounding medium is at least 3.5;
b) an outer coating surrounding each enterically coated particle or one or more outer coatings surrounding a plurality of particles, wherein:
i) said outer coating comprises a sufficient amount of an acid inhibitor to suppress gastric acid secretion within 6 hours after ingestion by a patient, and wherein said acid inhibitor is selected from the group consisting of: a proton pump inhibitor; and an H2 blocker;
ii) said outer coating is not an enteric coating and is not surrounded by an enteric coating.

13. The pharmaceutical composition of claim 12, wherein said outer coating:
a) has a thickness of less than 1000 microns; or
b) comprises a stabilizer or a buffer; or
c) both has a thickness of less than 1000 microns and comprises a stabilizer or a buffer.

14. The pharmaceutical composition of claim 12, wherein said acid inhibitor in said outer coating is a proton pump inhibitor at 1-200 mg and is selected from the group consisting of: omeprazole; esomeprazole; lansoprazole; pantoprazole; and rabeprazole.

15. The pharmaceutical composition claim 12, wherein said acid inhibitor is a proton pump inhibitor selected from the group consisting of: AZD-0865, AR-H047108, CS-526, pumaprazole, revaprazan and soraprazan.

16. The pharmaceutical composition of claim 12, wherein said enterically coated core comprises 1-600 mg of said proton pump inhibitor, and wherein said proton pump inhibitor is selected from the group consisting of: omeprazole; esomeprazole; lansoprazole; pantoprazole; and rabeprazole.

17. The pharmaceutical composition of claim 16, wherein said proton pump inhibitor is selected from the group consisting of: omeprazole, present in said enterically coated core at between 5 mg and 50 mg; esomeprazole, present in said enterically coated core at 5-100 mg; lansoprazole, present in said enterically coated core at 15-150 mg; pantoprazole, present in said enterically coated core at between 10 mg and 200 mg; and rabeprazole, present in said enterically coated core at between 5 mg and 100 mg.

18. The pharmaceutical composition of claim 12, wherein:
a) said acid inhibitor in said outer coating is a proton pump inhibitor present at 1-200 mg and selected from the group consisting of: omeprazole; esomeprazole; lansoprazole; pantoprazole; rabeprazole; AZD-0865, AR-H047108, CS-526, pumaprazole, revaprazan and soraprazan; and
b) said enterically coated core comprises 5-600 mg of said proton pump inhibitor, and wherein said proton pump inhibitor is selected from the group consisting of: omeprazole; esomeprazole; lansoprazole; pantoprazole; and rabeprazole.

19. The pharmaceutical composition of claim 12, wherein said acid inhibitor in said outer coating is an H2 blocker.

20. The pharmaceutical composition of claim 19, wherein said acid inhibitor is said acid inhibitor is present in said outer coating at 1-300 mg and is selected from the group consisting of: cimetidine; ranitidine; famotidine; ebrotidine; pabutidine; lafutidine; and nizatidine.

21. The pharmaceutical composition of claim 19, wherein said enterically coated core comprises 5-600 mg of said proton pump inhibitor, and wherein said proton pump inhibitor is selected from the group consisting of: omeprazole; esomeprazole; lansoprazole; pantoprazole; and rabeprazole.

22. The pharmaceutical composition of claim 21, wherein said proton pump inhibitor is selected from the group consisting of: omeprazole, present in said enterically coated core at between 5 mg and 50 mg; esomeprazole, present in said enterically coated core at 5-100 mg; lansoprazole, present in said enterically coated core at 15-150 mg; pantoprazole, present in said enterically coated core at between 10 mg and 200 mg; and rabeprazole, present in said enterically coated core at between 5 mg and 100 mg.

23. The pharmaceutical composition of claim 19, wherein:
a) said acid inhibitor in said outer coating is an H2 blocker present at 1-300 mg and selected from the group consisting of: cimetidine; ranitidine; famotidine;
ebrotidine; pabutidine; lafutidine; and nizatidine; and
b) said enterically coated core comprises 5-600 mg of said proton pump inhibitor, and wherein said proton pump inhibitor is selected from the group consisting of: omeprazole, esomeprazole, lansoprazole, pantoprazole and rabeprazole.

24. A method of treating a patient for a disease or condition characterized by abnormal gastric acid production, gastric acid reflux or damage to the gastrointestinal tract comprising administering to said patient the pharmaceutical composition of claim 1.

25. A method of treating a patient for a disease or condition characterized by abnormal gastric acid production, gastric acid reflux or damage to the gastrointestinal tract comprising administering to said patient the pharmaceutical composition of claim 12.

26. A method of manufacturing a tablet unit dosage form or a coated drug pellet for inclusion in a tablet or capsule, said method comprising:
a) forming a core comprising 5-600 mg of a proton pump inhibitor;
(b) applying an enteric coating to said core; and
(c) spraying an outer coating over said enteric coating, wherein said outer coating:
i) is not enteric; and
ii) comprises either 1-200 mg of a proton pump inhibitor or 1-300 mg of an H2 blocker.

26. The method claim 26, wherein said tablet or coated drug pellet has at least one characteristic selected from the group consisting of:
a) said outer coating has a thickness of less than 1000 microns;
b) said outer coating further comprises a stabilizer or a buffer;
c) said enteric coating is pH sensitive and does not release the proton pump inhibitor in said core until the surrounding pH is 5.5 or higher;
d) said acid inhibitor in said outer coating is selected from the group consisting of: omeprazole; esomeprazole; lansoprazole; pantoprazole; rabeprazole; AZD-0865, AR-H047108, CS-526, pumaprazole, revaprazan and soraprazan;
e) said acid inhibitor in said outer coating is an H2 blocker selected from the group consisting of: cimetidine; ranitidine; famotidine; ebrotidine; pabutidine; lafutidine; and nizatidine.
